(19) United States
(12) Patent Application Publication
(10) Pub. No.: US 2006/0282065 A1
(43) Pub. Date: Dec. 14, 2006

(54) Microtools and methods for fabricating such tools

(75) Inventor: Adam L. Cohen, Van Nuys, CA (US)

Correspondence Address: MICROFABRICA INC.;ATT: DENNIS R. SMALLEY, 7911 HASKELL AVENUE, VAN NUYS, CA, 91406, US

(73) Assignee: Microfabrica, Inc.

(21) Appl. No.: 11/444,999

(22) Filed: May 31, 2006

Related U.S. Application Data
(63) Continuation-in-part of application No. 10/697,598, filed on Oct. 29, 2003, now abandoned.
(63) Continuation-in-part of application No. 10/697,598, filed on Oct. 29, 2003, now abandoned.
(60) Provisional application No. 60/686,496, filed on May 31, 2005.
(60) Provisional application No. 60/422,007, filed on Oct. 29, 2002.

(51) Int. Cl. A61B 17/29

(52) U.S. Cl. 606/1; 606/207

(57) ABSTRACT

Embodiments of the present invention provide mesoscale or microscale three-dimensional devices, structures, instruments, and the like. In particular, instruments that are useable in minimally invasive surgery are described that include multiple tools that are deployable from a distal end of one or more housings or retractable into a distal end of one or more housings via the applying of tension to either end of one or more chain or chain-like elements that extend from the proximal end of the one or more housings.

### RELATED APPLICATIONS

This application claims benefit of U.S. Provisional Patent Application No. 60/686,496, filed May 31, 2005 and is a continuation-in-part of U.S. patent application Ser. No. 10/697,598, filed Oct. 29, 2003, which in turn claims benefit of U.S. Provisional Patent Application No. 60/422,007, filed Oct. 29, 2002. Each of these applications is hereby incorporated herein by reference as if set forth in full herein.

### FIELD OF THE INVENTION

Certain embodiments of the present invention relate to the field of electrochemical fabrication and the associated formation of three-dimensional structures, instruments, or devices formed a from a plurality of adhered layers with each layer formed from at least one structural material and at least one sacrificial material (e.g. microscale or mesoscale structures). Particular embodiments relate to microscale or mesoscale structures, instruments, or devices that may be useable in surgical procedures and in particular to minimally invasive surgical procedures.

### BACKGROUND OF THE INVENTION

A technique for forming three-dimensional structures (e.g. parts, components, devices, and the like) from a plurality of adhered layers was invented by Adam L. Cohen and is known as Electrochemical Fabrication. It is being commercially pursued by Microfabrica® Inc. (formerly MEMGen® Corporation) of Burbank, Calif. under the name EFAB®. Certain variations of this technique were described in U.S. Pat. No. 6,027,630, issued on Feb. 22, 2000. The disclosed electrochemical deposition techniques allow the selective deposition of a material using a unique masking technique that involves the use of a mask that includes patterned conformable material on a support structure that is independent of the substrate onto which plating will occur. When desiring to perform an electrodeposition using the mask, the conformable portion of the mask is brought into contact with a substrate while in the presence of a plating solution such that the contact of the conformable portion of the mask to the substrate inhibits deposition at selected locations. For convenience, these masks might be generically called conformable contact masks; the masking technique may be generically called a conformable contact mask plating process. More specifically, in the terminology of Microfabrica Inc. (formerly MEMGen® Corporation) of Burbank, Calif. such masks have come to be known as INSTANT MASKS™ and the process known as INSTANT MASKING™ or INSTANT MASKT™ plating. Selective depositions using conformable contact mask plating may be used to form single layers of material or may be used to form multi-layer structures. The teachings of the '630 patent are hereby incorporated herein by reference as if set forth in full herein. Since the filing of the patent application that led to the above noted patent, various papers about conformable contact mask plating (i.e. INSTANT MASKING) and electrochemical fabrication have been published:
(1) A. Cohen, G. Zhang, F. Tseng, F. Mansfeld, U. Frodis and P. Will, “EFAB: Batch production of functional, fully-dense metal parts with micro-scale features”, Proc. 9th Solid Freeform Fabrication, The University of Texas at Austin, p161, August 1998.
(2) A. Cohen, G. Zhang, F. Tseng, F. Mansfeld, U. Frodis and P. Will, “EFAB: Rapid, Low-Cost Desktop Micromachining of High Aspect Ratio True 3-D MEMS”, Proc. 12th IEEE Micro Electro Mechanical Systems Workshop, IEEE, p244, January 1999.
(3) A. Cohen, “3-D Micromachining by Electrochemical Fabrication”, Micromachine Devices, March 1999.
(4) G. Zhang, A. Cohen, U. Frodis, F. Tseng, F. Mansfeld, and P. Will, “EFAB: Rapid Desktop Manufacturing of True 3-D Microstructures”, Proc. 2nd International Conference on Integrated MicroNanotechnology for Space Applications, The Aerospace Co., April 1999.
(5) F. Tseng, U. Frodis, G. Zhang, A. Cohen, F. Mansfeld, and P. Will, “EFAB: High Aspect Ratio, Arbitrary 3-D Metal Microstructures using a Low-Cost Automated Batch Process”, 3rd International Workshop on High Aspect Ratio MicroStructure Technology (HARMST'99), June 1999.
(6) A. Cohen, U. Frodis, F. Tseng, G. Zhang, F. Mansfeld, and P. Will, “EFAB: Low-Cost, Automated Electrochemical Batch Fabrication of Arbitrary 3-D Microstructures”, Micromachining and Microfabrication Process Technology, SPIE 1999 Symposium on Micromachining and Microfabrication, September 1999.
(7) F. Tseng, G. Zhang, U. Frodis, A. Cohen, F. Mansfeld, and P. Will, “EFAB: High Aspect Ratio, Arbitrary 3-D Metal Microstructures using a Low-Cost Automated Batch Process”, MEMS Symposium, ASME 1999 International Mechanical Engineering Congress and Exposition, November, 1999.
(8) A. Cohen, “Electrochemical Fabrication (EFABTM)”, Chapter 19 of The MEMS Handbook, edited by Mohamed Gad-EI-Hak, CRC Press, 2002.
(9) Microfabrication-Rapid Prototyping's Killer Application”, pages 1-5 of the Rapid Prototyping Report, CAD/CAM Publishing, Inc., June 1999.

The disclosures of these nine publications are hereby incorporated herein by reference as if set forth in full herein.

The electrochemical deposition process may be carried out in a number of different ways as set forth in the above patent and publications. In one form, this process involves the execution of three separate operations during the formation of each layer of the structure that is to be formed:
1. Selectively depositing at least one material by electrodeposition upon one or more desired regions of a substrate.
2. Then, blanket depositing at least one additional material by electrodeposition so that the additional deposit covers both the regions that were previously selectively deposited onto, and the regions of the substrate that did not receive any previously applied selective depositions.
3. Finally, planarizing the materials deposited during the first and second operations to produce a smoothed surface of a first layer of desired thickness having at least one region containing the at least one material and at least one region containing at least the one additional material.

After formation of the first layer, one or more additional layers may be formed adjacent to the immediately preceding layer and adhered to the smoothed surface of that preceding layer. These additional layers are formed by repeating the first through third operations one or more times wherein the formation of each subsequent layer treats the previously formed layers and the initial substrate as a new and thickening substrate.

Once the formation of all layers has been completed, at least a portion of at least one of the materials deposited is generally removed by an etching process to expose or release the three-dimensional structure that was intended to be formed.

The preferred method of performing the selective electrodeposition involved in the first operation is by conformable contact mask plating. In this type of plating, one or more conformable contact (CC) masks are first formed. The CC masks include a support structure onto which a patterned conformable dielectric material is adhered or formed. The conformable material for each mask is shaped in accordance with a particular cross-section of material to be plated. At least one CC mask is needed for each unique cross-sectional pattern that is to be plated.

The support for a CC mask is typically a plate-like structure formed of a metal that is to be selectively electroplated and from which material to be plated will be dissolved. In this typical approach, the support will act as an anode in an electroplating process. In an alternative approach, the support may instead be a porous or otherwise perforated material through which deposition material will pass during an electroplating operation on its way from a distal anode to a deposition surface. In either approach, it is possible for CC masks to share a common support, i.e. the patterns of conformable dielectric material for plating multiple layers of material may be located in different areas of a single support structure. When a single support structure contains multiple plating patterns, the entire structure is referred to as the CC mask while the individual plating masks may be referred to as “submasks”. In the present application such a distinction will be made only when relevant to a specific point being made.

In preparation for performing the selective deposition of the first operation, the conformable portion of the CC mask is placed in registration with and pressed against a selected portion of the substrate (or onto a previously formed layer or onto a previously deposited portion of a layer) on which deposition is to occur. The pressing together of the CC mask and substrate occur in such a way that all openings, in the conformable portions of the CC mask contain plating solution. The conformable material of the CC mask that contacts the substrate acts as a barrier to electrodeposition while the openings in the CC mask that are filled with electroplating solution act as pathways for transferring material from an anode (e.g. the CC mask support) to the non-contacted portions of the substrate (which act as a cathode during the plating operation) when an appropriate potential and/or current are supplied.

An example of a CC mask and CC mask plating are shown in FIGS. 1A-1C. FIG. 1A shows a side view of a CC mask 8 consisting of a conformable or deformable (e.g. elastomeric) insulator 10 patterned on an anode 12. The anode has two functions. One is as a supporting material for the patterned insulator 10 to maintain its integrity and alignment since the pattern may be topologically complex (e.g., involving isolated “islands” of insulator material). The other function is as an anode for the electroplating operation. FIG. 1A also depicts a substrate 6 separated from mask 8. CC mask plating selectively deposits material 22 onto a substrate 6 by simply pressing the insulator against the substrate then electrodepositing material through apertures 26a and 26b in the insulator as shown in FIG. 1B. After deposition, the CC mask is separated, preferably non-destructively, from the substrate 6 as shown in FIG. 1C. The CC mask plating process is distinct from a “through-mask” plating process in that in a through-mask plating process the separation of the masking material from the substrate would occur destructively. As with through-mask plating, CC mask plating deposits material selectively and simultaneously over the entire layer. The plated region may consist of one or more isolated plating regions where these isolated plating regions may belong to a single structure that is being formed or may belong to multiple structures that are being formed simultaneously. In CC mask plating as individual masks are not intentionally destroyed in the removal process, they may be usable in multiple plating operations.

Another example of a CC mask and CC mask plating is shown in FIGS. 1D-1G. FIG. 1D shows an anode 12′ separated from a mask 8′ that includes a patterned conformable material 10′ and a support structure 20. FIG. 1D also depicts substrate 6 separated from the mask 8′. FIG. 1E illustrates the mask 8′ being brought into contact with the substrate 6. FIG. 1F illustrates the deposit 22′ that results from conducting a current from the anode 12′ to the substrate 6. FIG. 1G illustrates the deposit 22′ on substrate 6 after separation from mask 8′. In this example, an appropriate electrolyte is located between the substrate 6 and the anode 12′ and a current of ions coming from one or both of the solution and the anode are conducted through the opening in the mask to the substrate where material is deposited. This type of mask may be referred to as an anodeless INSTANT MASK™ (AIM) or as an anodeless conformable contact (ACC) mask.

Unlike through-mask plating, CC mask plating allows CC masks to be formed completely separate from the fabrication of the substrate on which plating is to occur (e.g. separate from a three-dimensional (3D) structure that is being formed). CC masks may be formed in a variety of ways, for example, a photolithographic process may be used. All masks can be generated simultaneously, e.g. prior to structure fabrication rather than during it. This separation makes possible a simple, low-cost, automated, self-contained, and internally-clean “desktop factory” that can be installed almost anywhere to fabricate 3D structures, leaving any required clean room processes, such as photolithography to be performed by service bureaus or the like.

An example of the electrochemical fabrication process discussed above is illustrated in FIGS. 2A-2F. These figures show that the process involves deposition of a first material 2 which is a sacrificial material and a second material 4 which is a structural material. The CC mask 8, in this example, includes a patterned conformable material (e.g. an elastomeric dielectric material) 10 and a support 12 which is made from deposition material 2. The conformal portion of the CC mask is pressed against substrate 6 with a plating solution 14 located within the openings 16 in the conformable material 10. An electric current, from power supply 18, is then passed through the plating solution 14 via (a) support 12 which doubles as an anode and (b) substrate 6 which doubles as a cathode. FIG. 2A illustrates that the passing of current causes material 2 within the plating solution and material 2 from the anode 12 to be selectively transferred to and plated on the substrate 6. After electroplating the first deposition material 2 onto the substrate 6 using CC mask 8, the CC mask 8 is removed as shown in FIG. 2B. FIG. 2C depicts the second deposition material 4 as having been blanket-deposited (i.e. non-selectively deposited) over the previously deposited first deposition material 2 as well as over the other portions of the substrate 6. The blanket deposition occurs by electroplating from an anode (not shown), composed of the second material, through an appropriate plating solution (not shown), and to the cathode/substrate 6. The entire two-material layer is then planarized to achieve precise thickness and flatness as shown in FIG. 2D. After repetition of this process for all layers, the multi-layer structure 20 formed of the second material 4 (i.e. structural material) is embedded in first material 2 (i.e. sacrificial material) as shown in FIG. 2E. The embedded structure is etched to yield the desired device, i.e. structure 20, as shown in FIG. 2F.

Various components of an exemplary manual electrochemical fabrication system 32 are shown in FIGS. 3A-3C. The system 32 consists of several subsystems 34, 36, 38, and 40. The substrate holding subsystem 34 is depicted in the upper portions of each of FIGS. 3A-3C and includes several components: (1) a carrier 48, (2) a metal substrate 6 onto which the layers are deposited, and (3) a linear slide 42 capable of moving the substrate 6 up and down relative to the carrier 48 in response to drive force from actuator 44. Subsystem 34 also includes an indicator 46 for measuring differences in vertical position of the substrate which may be used in setting or determining layer thicknesses and/or deposition thicknesses. The subsystem 34 further includes feet 68 for carrier 48 which can be precisely mounted on subsystem 36.

The CC mask subsystem 36 shown in the lower portion of FIG. 3A includes several components: (1) a CC mask 8 that is actually made up of a number of CC masks (i.e. submasks) that share a common support/anode 12, (2) precision X-stage 54, (3) precision Y-stage 56, (4) frame 72 on which the feet 68 of subsystem 34 can mount, and (5) a tank 58 for containing the electrolyte 16. Subsystems 34 and 36 also include appropriate electrical connections (not shown) for connecting to an appropriate power source (not shown) for driving the CC masking process.

The blanket deposition subsystem 38 is shown in the lower portion of FIG. 3B and includes several components: (1) an anode 62, (2) an electrolyte tank 64 for holding plating solution 66, and (3) frame 74 on which feet 68 of subsystem 34 may sit. Subsystem 38 also includes appropriate electrical connections (not shown) for connecting the anode to an appropriate power supply (not shown) for driving the blanket deposition process.

The planarization subsystem 40 is shown in the lower portion of FIG. 3C and includes a lapping plate 52 and associated motion and control systems (not shown) for planarizing the depositions.

In addition to teaching the use of CC masks for electrodeposition purposes, the '630 patent also teaches that the CC masks may be placed against a substrate with the polarity of the voltage reversed and material may thereby be selectively removed from the substrate. It indicates that such removal processes can be used to selectively etch, engrave, and polish a substrate, e.g., a plaque.

The '630 patent further indicates that the electroplating methods and articles disclosed therein allow fabrication of devices from thin layers of materials such as, e.g., metals, polymers, ceramics, and semiconductor materials. It further indicates that although the electroplating embodiments described therein have been described with respect to the use of two metals, a variety of materials, e.g., polymers, ceramics and semiconductor materials, and any number of metals can be deposited either by the electroplating methods therein, or in separate processes that occur throughout the electroplating method. It indicates that a thin plating base can be deposited, e.g., by sputtering, over a deposit that is insufficiently conductive (e.g., an insulating layer) so as to enable subsequent electroplating. It also indicates that multiple support materials (i.e. sacrificial materials) can be included in the electroplated element allowing selective removal of the support materials.

The '630 patent additionally teaches that the electroplating methods disclosed therein can be used to manufacture elements having complex microstructure and close tolerances between parts. An example is given with the aid of FIGS. 14A-14E of that patent. In the example, elements having parts that fit with close tolerances, e.g., having gaps between about 1-5 um, including electroplating the parts of the device in an unassembled, preferably pre-aligned, state and once fabricated. In such embodiments, the individual parts can be moved into operational relation with each other or they can simply fall together. Once together the separate parts may be retained by clips or the like.

Another method for forming microstructures from electroplated metals (i.e. using electrochemical fabrication techniques) is taught in U.S. Pat. No. 5,190,637 to Henry Guckel, entitled “Formation of Microstructures by Multiple Level Deep X-ray Lithography with Sacrificial Metal layers”. This patent teaches the formation of metal structure utilizing mask exposures. A first layer of a primary metal is electroplated onto an exposed plating base to fill a void in a photoresist, the photoresist is then removed and a secondary metal is electroplated over the first layer and over the plating base. The exposed surface of the secondary metal is then machined down to a height which exposes the first metal to produce a flat uniform surface extending across the both the primary and secondary metals. Formation of a second layer may then begin by applying a photoresist layer over the first layer and then repeating the process used to produce the first layer. The process is then repeated until the entire structure is formed and the secondary metal is removed by etching. The photoresist is formed over the plating base or previous layer by casting and the voids in the photoresist are formed by exposure of the photoresist through a patterned mask via X-rays or UV radiation.

The '637 patent teaches the locating of a plating base onto a substrate in preparation for electroplating materials onto the substrate. The plating base is indicated as typically involving the use of a sputtered film of an adhesive metal, such as chromium or titanium, and then a sputtered film of the metal that is to be plated. It is also taught that the plating base may be applied over an initial sacrificial layer of material on the substrate so that the structure and substrate may be detached if desired. In such cases after formation of the structure the plating base may be patterned and removed from around the structure and then the sacrificial layer under the plating base may be dissolved to free the structure. Substrate materials mentioned in the '637 patent include silicon, glass, metals, and silicon with protected processed semiconductor devices. A specific example of a plating base includes about 150 angstroms of titanium and about 300 angstroms of nickel, both of which are sputtered at a temperature of 160° C. In another example it is indicated that the plating base may consist of 150 angstroms of titanium and 150 angstroms of nickel where both are applied by sputtering.

### SUMMARY OF THE INVENTION

It is an object of some aspects of the invention to provide improved micro or mesoscale tools or instruments.

It is an object of some aspects of the invention to provide improved micro or mesoscale multi-functional tools or instruments.

It is an object of some aspects of the invention to provide improved micro or mesoscale tools or instruments for minimally invasive surgery.

It is an object of some aspects of the invention to provide improved micro or mesoscale multi-functional tools or instruments for minimally invasive surgery.

It is an object of some aspects of the invention to provide micro or mesoscale tools or instruments for minimally invasive surgery where interactive portions of the tool or instrument is extended from a distal end of a housing by exerting tension on a proximal end of a sheath (e.g. a catheter) that extends from a distal end of an instrument housing.

It is an object of other aspects of the invention to provide methods for fabricating tools or instruments that provide the above noted objects of the invention as well as tools or instruments that meet other objects of the invention.

Other objects and advantages of various embodiments of the invention will be apparent to those of skill in the art upon review of the teachings herein. The various embodiments of the invention, set forth explicitly herein or otherwise ascertained from the teachings herein, may address one or more of the above objects alone or in combination, or alternatively they may address some other object of the invention that may be ascertained from the teachings herein. It is not intended that all objects be addressed by any single aspect of the invention even though that may be the case with regard to some aspects.

In a first aspect of the invention a micro-scale or mesoscale instrument containing multiple independently deployable tools including: a first tool having a first functionality; a second tool having a second functionality; at least one housing, having at least one proximal end and at least one distal end, for holding the first and second tools when in a retracted state a housing; at least one mechanism extending from the proximal end of the at least one housing to which a tensional force may be exerted to cause deployment of at least one of the first or second tool from the at least one housing.

In a second aspect of the invention a micro-scale or mesoscale surgical instrument containing multiple independently deployable tools including: a first module including a first housing and a first tool having a first functionality which is deployable from the first housing; a second module including a second housing and a second tool having a second functionality which is deployable form the second housing, wherein the first module and second module are stacked and fixed together.

In a third aspect of the invention a micro-scale or mesoscale instrument, including: a module including a housing and a tool having a first functionality which is deployable from a distal end of the housing; a bidirectional chain connected to the tool within the housing and having two ends that extend from the housing, wherein pulling on a first end of the chain causes a retracted tool to be deployed from the housing; wherein pulling on a second end of the chain, which is different from the first end, causes a deployed tool to be retracted into the housing.

In a fourth aspect of the invention a micro-scale or mesoscale instrument, including: a module including a housing and a tool having a first functionality which is deployable from a distal end of the housing; a mechanism for deploying the tool from the housing, wherein the tool is deployable from the housing along a non-pivoting path.

Further aspects of the invention will be understood by those of skill in the art upon reviewing the teachings herein. Other aspects of the invention may involve combinations of the above noted aspects of the invention. These other aspects of the invention may provide various combinations of the aspects, embodiments, and associated alternatives explicitly set forth herein as well as provide other configurations, structures, functional relationships, and processes that have not been specifically set forth above.

### BRIEF DESCRIPTION OF THE DRAWINGS

FIGS. 1A-1C schematically depict side views of various stages of a CC mask plating process, while FIGS. 1D-1G schematically depict a side views of various stages of a CC mask plating process using a different type of CC mask.

FIGS. 2A-2F schematically depict side views of various stages of an electrochemical fabrication process as applied to the formation of a particular structure where a sacrificial material is selectively deposited while a structural material is blanket deposited.

FIGS. 3A-3C schematically depict side views of various example subassemblies that may be used in manually implementing the electrochemical fabrication method depicted in FIGS. 2A-2F.

FIGS. 4A-4F schematically depict the formation of a first layer of a structure using adhered mask plating where the blanket deposition of a second material overlays both the openings between deposition locations of a first material and the first material itself

FIG. 4G depicts the completion of formation of the first layer resulting from planarizing the deposited materials to a desired level.

FIGS. 4H and 4I respectively depict the state of the process after formation of the multiple layers of the structure and after release of the structure from the sacrificial material.

FIG. 5 provides a top perspective view of a two tool instrument according an embodiment of the invention.

FIG. 6 provides a bottom perspective view of the two tool instrument of FIG. 5.

FIGS. 7-10 provide various micrographs of a two tool instrument, similar to that of FIGS. 5 and 6, as fabricated using an electrochemical fabrication method.

FIG. 11 provides a bottom perspective close up view of the proximal end of the instrument of FIGS. 5 and 6

FIG. 12 provides a bottom perspective close up view of the distal end of the instrument of FIGS. 5 and 6.

FIG. 13 provides a close up view of a portion of a chain as used in the instrument of FIGS. 5 and 6.

FIG. 14 provides a perspective sectional view of distal end of the lower chamber of the upper module of the instrument of FIGS. 5 and 6.

FIG. 15 provides a perspective sectional view from the proximal side of the lower module without its upper lid so that the tool within the upper chamber of upper compartment of the lower module of FIGS. 5 and 6 may be seen.

FIG. 16 provides a close up of a portion of FIG. 15 so that the attachment between the tool and the chain of the lower module of FIGS. 5 and 6 may be seen.

FIG. 17 provides a perspective sectional view of the upper chamber of the upper module of the instrument of FIGS. 5 and 6 without the lid of the upper chamber so that the tool within the chamber may be seen.

FIG. 18 provides a close up of a portion of FIG. 17 so that the jaws and various details of the forceps tool in the upper module can be seen.

### DETAILED DESCRIPTION OF PREFERRED EMBODIMENTS OF THE INVENTION

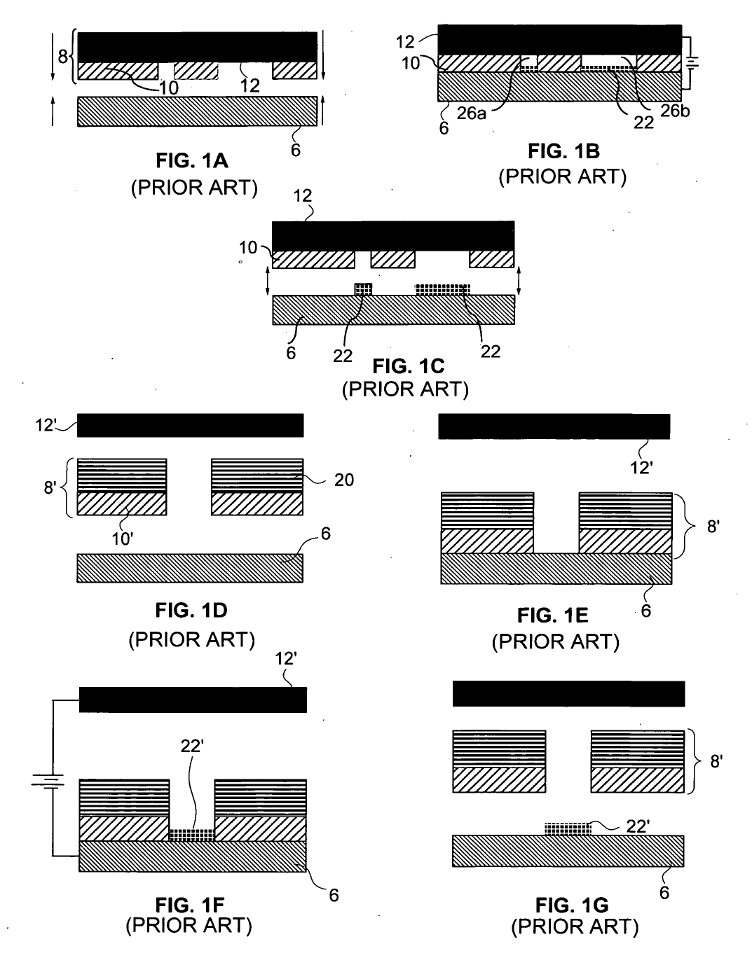

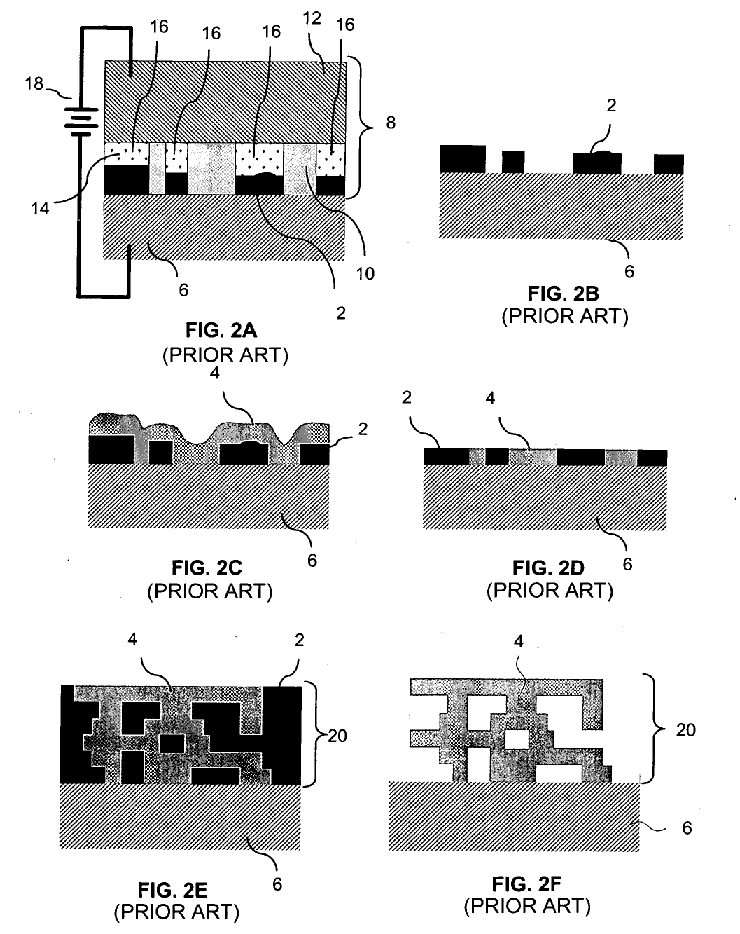

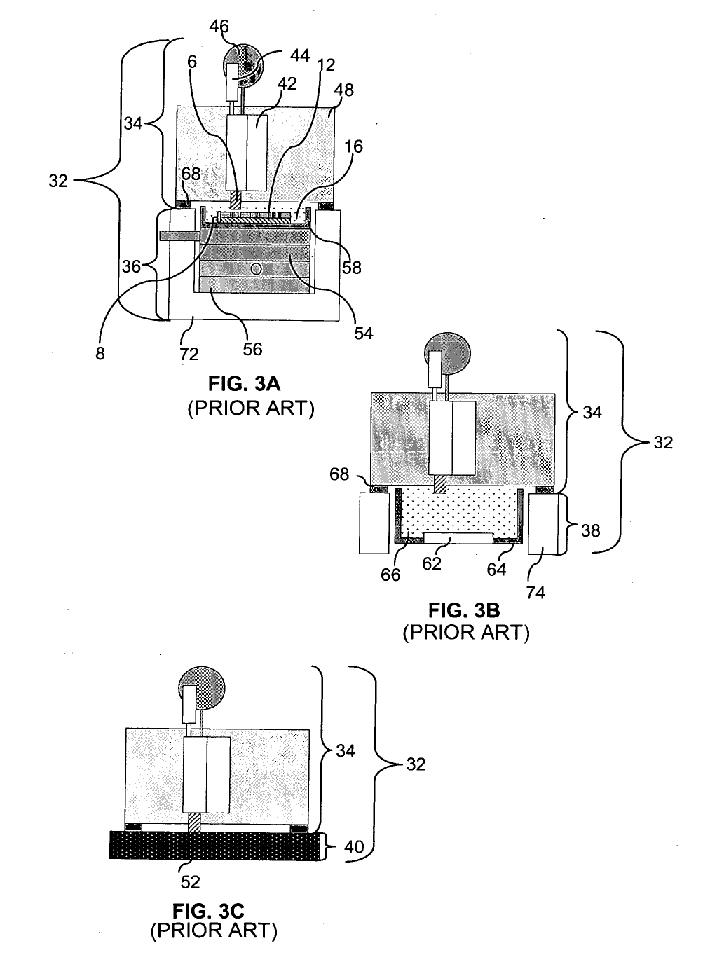

FIGS. 1A-1G, 2A-2F, and 3A-3C illustrate various features of one form of electrochemical fabrication that are known. Other electrochemical fabrication techniques are set forth in the '630 patent referenced above, in the various previously incorporated publications, in various other patents and patent applications incorporated herein by reference, still others may be derived from combinations of various approaches described in these publications, patents, and applications, or are otherwise known or ascertainable by those of skill in the art from the teachings set forth herein. All of these techniques may be combined with those of the various embodiments of various aspects of the invention to yield enhanced embodiments. Still other embodiments may be derived from combinations of the various embodiments explicitly set forth herein.

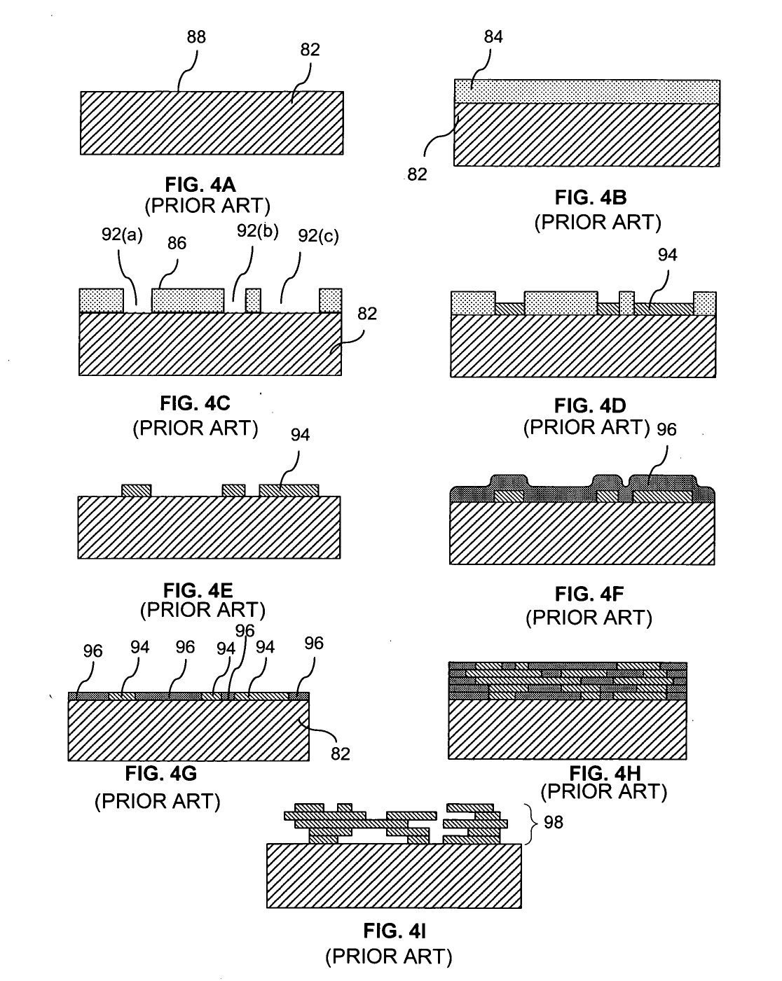

FIGS. 4A-4I illustrate various stages in the formation of a single layer of a multi-layer fabrication process where a second metal is deposited on a first metal as well as in openings in the first metal so that the first and second metal form part of the layer. In FIG. 4A a side view of a substrate 82 is shown, onto which patternable photoresist 84 is cast as shown in FIG. 4B. In FIG. 4C, a pattern of resist is shown that results from the curing, exposing, and developing of the resist. The patterning of the photoresist 84 results in openings or apertures 92(a)-92(c) extending from a surface 86 of the photoresist through the thickness of the photoresist to surface 88 of the substrate 82. In FIG. 4D a metal 94 (e.g. nickel) is shown as having been electroplated into the openings 92(a)-92(c). In FIG. 4E the photoresist has been removed (i.e. chemically stripped) from the substrate to expose regions of the substrate 82 which are not covered with the first metal 94. In FIG. 4F a second metal 96 (e.g. silver) is shown as having been blanket electroplated over the entire exposed portions of the substrate 82 (which is conductive) and over the first metal 94 (which is also conductive). FIG. 4G depicts the completed first layer of the structure which has resulted from the planarization of the first and second metals down to a height that exposes the first metal and sets a thickness for the first layer. In FIG. 4H the result of repeating the process steps shown in FIGS. 4B-4G several times to form a multi-layer structure are shown where each layer consists of two materials. For most applications, one of these materials is removed as shown in FIG. 4I to yield a desired 3-D structure 98 (e.g. component or device).

Various embodiments of various aspects of the invention are directed to formation of three-dimensional structures from materials some of which may be electrodeposited or electroless deposited. Some of these structures may be formed form a single layer of one or more deposited material while others are formed from a plurality of layers each including at least two materials (e.g. two or more layers, more preferably five or more layers, and most preferably ten or more layers). In some embodiments microscale structures are produced that have features positioned with micron or near micron-level precision and with minimum features size on the order of microns to tens of microns. In other embodiments mesoscale structures with less precise feature placement (tens to hundreds of microns) and/or larger minimum features (tens to hundreds of microns) may be formed. In some embodiments microscale structures may have overall dimensions on the order of millimeters or even centimeters while in other embodiments the microstructures may have smaller overall dimensions. In some embodiments, mesoscale structures may have overall dimensions on the order of millimeters to centimeters while in other embodiments they may be smaller. In still other embodiments, microscale structures may have higher precision and smaller minimum feature sizes.

The various embodiments, alternatives, and techniques disclosed herein may form multi-layer structures using a single patterning technique on all layers or using different patterning techniques on different layers. For example, Various embodiments of the invention may perform selective patterning operations using conformable contact masks and masking operations (i.e. operations that use masks which are contacted to but not adhered to a substrate), proximity masks and masking operations (i.e. operations that use masks that at least partially selectively shield a substrate by their proximity to the substrate even if contact is not made), non-conformable masks and masking operations (i.e. masks and operations based on masks whose contact surfaces are not significantly conformable), and/or adhered masks and masking operations (masks and operations that use masks that are adhered to a substrate onto which selective deposition or etching is to occur as opposed to only being contacted to it). Adhered mask may be formed in a number of ways including (1) by application of a photoresist, selective exposure of the photoresist, and then development of the photoresist, (2) selective transfer of pre-patterned masking material, and/or (3) direct formation of masks from computer controlled depositions of material.

Patterning operations may be used in selectively depositing material and/or may be used in the selective etching of material. Selectively etched regions may be selectively filled in or filled in via blanket deposition, or the like, with a different desired material. In some embodiments, the layer-by-layer build up may involve the simultaneous formation of portions of multiple layers. In some embodiments, depositions made in association with some layer levels may result in depositions to regions associated with other layer levels. Such use of selective etching and interlaced material deposited in association with multiple layers is described in U.S. patent application Ser. No. 10/434,519, by Smalley, and entitled “Methods of and Apparatus for Electrochemically Fabricating Structures Via Interlaced Layers or Via Selective Etching and Filling of Voids” which is hereby incorporated herein by reference as if set forth in full.

Various embodiments of the invention relate to modular, multi-functional microfabricated instruments that have application, inter alia, in minimally-invasive surgery (MIS) and in other domains (e.g. inspection and repair of components in tubes or other hard to access locations. An feature of some embodiments of the invention is, in effect, to provide a ‘Swiss army knife’-type instrument in which multiple tools (e.g. as many as required for a particular surgery or portion of a surgery to be performed) are enclosed within a single or multiple component housing and can be independently exposed, extended, or deployed from their housing(s) and then enclosed again (e.g. retracted into) by the housing(s). For MIS applications, remote actuation of the instrument by a surgeon is typical. Since it is possible to pass cables, wires, or chains through a catheter and apply tension to these from outside the body, a preferred embodiment of the invention provides extension, retraction, and actuation of each tool using tension alone. In such embodiments, a tool housing or head may be located at a distal end of the catheter while the proximal end extends from the body of the patient. As needed, tools or instruments (e.g. clamps, hooks, knifes, scissors, needles, sensors, sample extractors, cameras, various sensors, and the like) may be deployed from the housing(s) or drawn back into the housing by exerting tension on cables, chains, wires, or the like that extend from the proximal end of the catheter. In some embodiments, the instrument may be fixed at the end of the catheter while in other embodiments, it may be extended to the end of the catheter via a flexible but not compressive lead wire or the like which is attached or otherwise abuts the proximal end of the instrument housing.

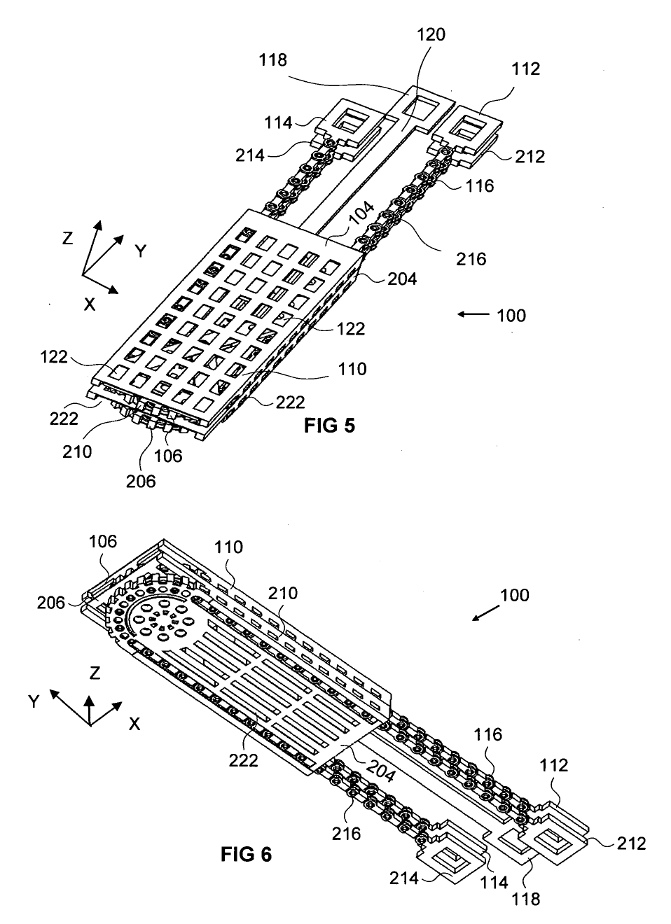

Since an MIS application (e.g., in a catheter or blood vessel) may not offer enough space for tools to swing out on pivots as in a Swiss army knife, in some embodiments of the invention (as illustrated herein with aid of FIGS. 5-18), the tools or instruments may extend through a telescopic motion along the main axis of the instrument (i.e. the axis of extending along the length of the catheter and housing, i.e. the y-axis in FIGS. 5-6) In some embodiments, to achieve extension and retraction through tension alone, a flexible chain is provided that wraps around a pulley located in the housing to change its direction by ˜180° (e.g. to provide two ends coming out the proximal end of the housing which may be attached to cables or chains extending along the length of the catheter). If the base or other portion of a tool or instrument is connected to one or more links on the chain, then pulling on one end of the chain will extend the tool while pulling on the other end will retract it. Other flexible structures such as wires and cables which can wrap around a pulley may also be used in addition to or in place of the chain. In the embodiments illustrated in FIGS. 5-18, multi-link chains are used since they eliminate any cyclic bending stress as compared with a single-piece structure.

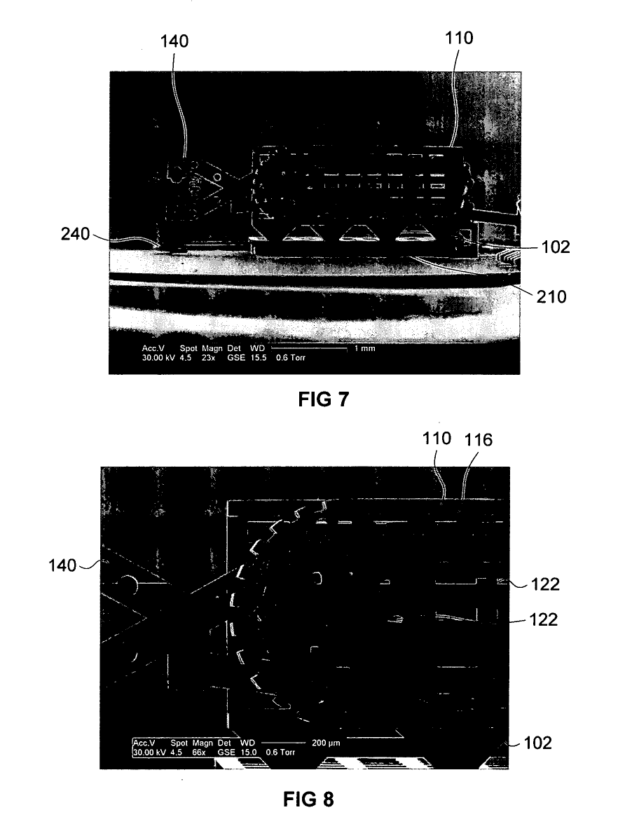

FIG. 5 shows a perspective view of the top side of an example multi-tool instrument 100 having two individual housings, an upper housing 110 and a lower housing 210. Right and left cables or chains 112 and 114 extend from the proximal end 104 of the upper housing while right and left cables or chains 212 and 214 extend from the proximal end 204 of the lower housing. FIG. 5 also shows an actuator tab 118 that connects to an actuator bar or arm 120 which extends from the proximal end 104 of the upper housing 110. The tab 118 may be grabbed directly or hooked to an extension cable or wire which may be moved to actuate a tool 140 (FIG. 7) after if is deployed from the upper housing 110. FIG. 5 also shows that the upper and lower housings include distal ends 106 and 206 respectively from which tools 140 and 240 can be deployed (FIG. 7). FIG. 6 provides a bottom side perspective view of the multi-tool instrument of FIG. 5.

In FIGS. 5 and 6 it can be seen that the upper and lower housings include numerous holes or openings 122 and 222 which may not be strictly necessary to achieve a desired functionality of the device but which instead may have been added to the design to make removal of sacrificial material easier. One or more sacrificial materials along with one or more structural materials may have been used during the fabrication of the device 100. Fabrication may be simplified using such sacrificial materials particularly when they the device 100 is formed from a plurality of adhered layers, e.g. by an electrochemical fabrication process such as one of those discussed herein above or discussed in various patent applications or other publications which are incorporated herein by reference In some such embodiments the structure may be formed from a plurality of layers each lying in an XY plane and each layer being stacked atop one another along the Z-axis. Of course in other embodiments other housing structures may be provided, including housings with smooth outer covers (e.g. on the sides and faces with the distal end exposed) and/or housings with smooth contours on the distal end which may help avoid damaging of tissues or interior portions of catheters during insertion or extraction.

FIGS. 7-10 show scanning electron microscope (SEM) micrographs of a similar instrument to that shown in FIGS. 5 and 6. The instrument of FIGS. 7-10 includes two housings with each holding its own tool. During formation, the two housings 110 and 210, and more specifically the two tool modules that include the two housings and associated tools) lay side-by-side with each housing connected to the other via a plurality of hinges 102. In FIGS. 7, 8, and 10 it can be seen that the upper housing 110 has been rotated ninety degrees relative to the lower housing. After rotating another ninety degrees the two housings 110 and 210 will lay back-to-back (as opposed to the back-to-front orientation of the devices shown in FIGS. 5 and 6. In the illustrated example, the hinge consists of a thin (e.g., 10 μm thick) plate that is composed of hourglass-shaped sections; when a folding force is applied to the un-anchored module, the hinge bends and plastically deforms at the ‘waist’ (i.e. the narrowest portion) of these sections. Folding is a potentially useful way to fabricate instruments of the sort contemplated here, if provision is made to retain the modules in the folded position against any spring back of the folded metal. In practice, a narrowed hinge may be used as an alternative to that shown here, to reduce overall width of the instrument. Building side-by-side and folding each offer an advantage to the fabrication of the overall instrument. Side-by-side building allows instrument formation to occur using fewer layers while folding provides some amount of alignment control during assembly. In some embodiments, after folding and secured attachment of separate modules, the hinges may be removed via mechanical cutting or laser ablation or the like so like as necessary precautions are taken to inhibit removed material from entering the modules and causing damage or jamming of tools. Such precautions may include embedding the structures in a temporary material (e.g. wax or the like) during hinge removal and thereafter removing the temporary material (e.g. via melting or dissolution).

In particular FIG. 7 provides a side micrograph view of the upper module 110 with its tool 140 (i.e. an example microscale forceps) in the deployed position along with a side view of the lower module 210 with its tool 240 (i.e. an example microscale hook) where the two modules are connected together via hinges 102.

FIG. 8 provide a close up micrograph view of the distal end of the upper module 110 such that the tool 140 can be seen extend from the distal end behind a cover element under which the chain 116 moves around a pulley. In FIG. 8 various openings 122 can also be seen.

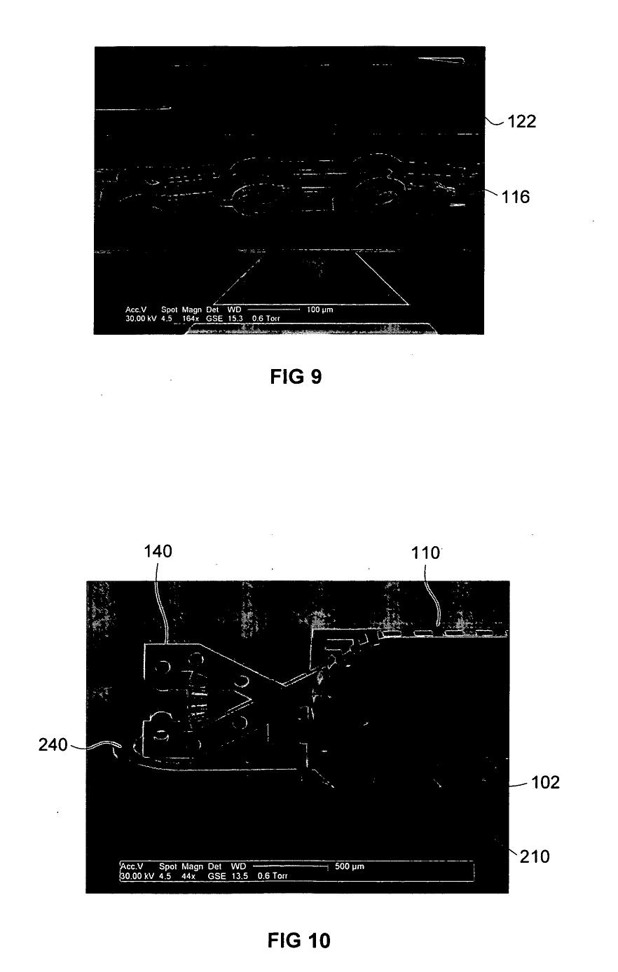

FIG. 9 provides a close up perspective micrograph view of a fabricated chain 116.

FIG. 10 provides a close up perspective micrograph view of the distal end of the both the upper and lower modules 110 and 210 which are oriented at ninety degrees to each other and where both tools 140 and 240 are deployed and visible,

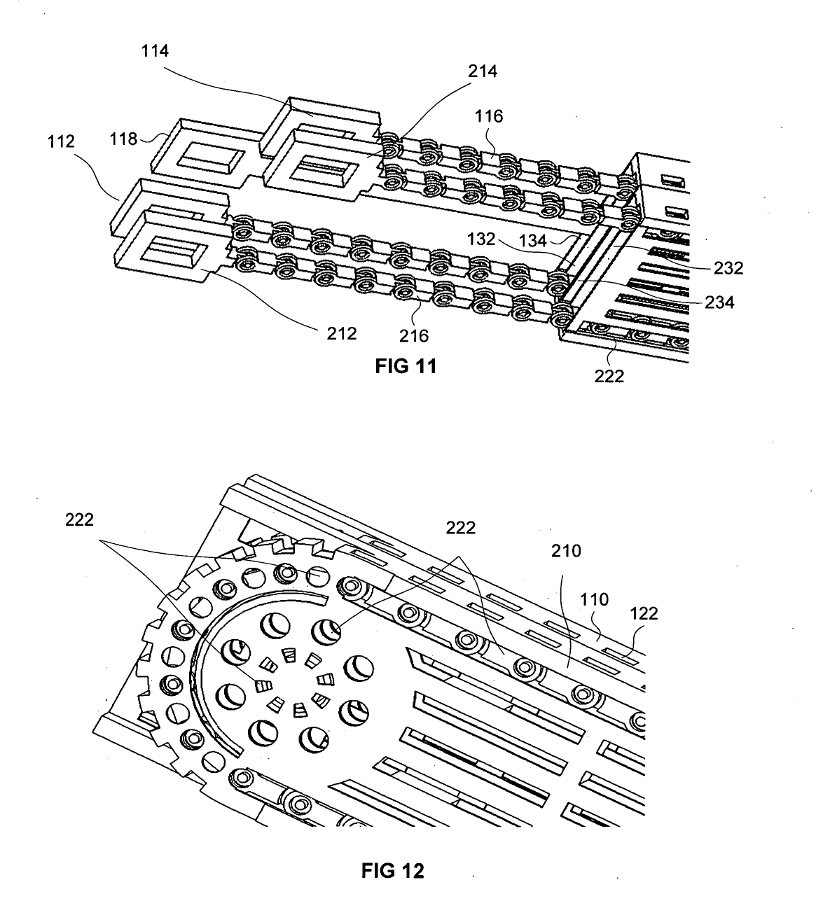

In some embodiments different pull tabs from those shown in FIGS. 5 and 6 are possible and may even be preferred in certain circumstances depending on the type of termination ultimately desired. The tabs shown are designed for testing purposes. A more compact termination that allows easy connection to a cable, wire, or chain may be preferable in a MIS application, for example. Each housing 110 and 210 or module is divided into upper and lower compartments 134 and 132 (for housing 110) and 234 and 232 as can be seen in FIG. 11. The upper compartments 134 and 234 in the embodiment shown, each house a respective tool when retracted, while the lower compartments 132 and 232 house the chain and pulley.

In the figures, an instrument 100 with two stacked modules is shown. In other embodiments, additional modules may be stacked, thus more complex instruments with greater functionality can be made. For example, an instrument consisting of four stacked modules can be fabricated that is ˜1 mm wide (X) and <1 mm in height (Z), since each module can be (and in this design, is) on the order of 200 μm tall. Of course in other embodiments other heights and width as well as height and width ratios may be implemented. Other ways of combing modules other than stacking can be used. Multiple modules may be combined and built as a single monolithic unit without assembly, or built separately and then assembled. The process of assembly may, for example, include using one or more of (1) fold over, (21) built-in alignment features, and/or (3) retention elements, such as clips, slide in mounts, adhesives, solder bonding, or the like. In some embodiments, final positioning may locate modules at angles relative to one another (i.e. non-parallel orientations).

FIG. 11 shows the pull chains 116 and 216 in more detail for a two-module instrument. Each module includes one pull chain passing over a pulley 226 (FIG. 14) such that both ends can be pulled. In the embodiment shown, the chain is long enough that both ends are always outside the housing of the module for accessibility and to allow for the large pull tabs shown.

FIG. 12 shows the bottom of the lower module 210 in detail with only the side of the upper module 110 being visible). The path of the chain 216 passing around the pulley 226 (FIG. 14) can be discerned through the release holes 222 in the module housing.

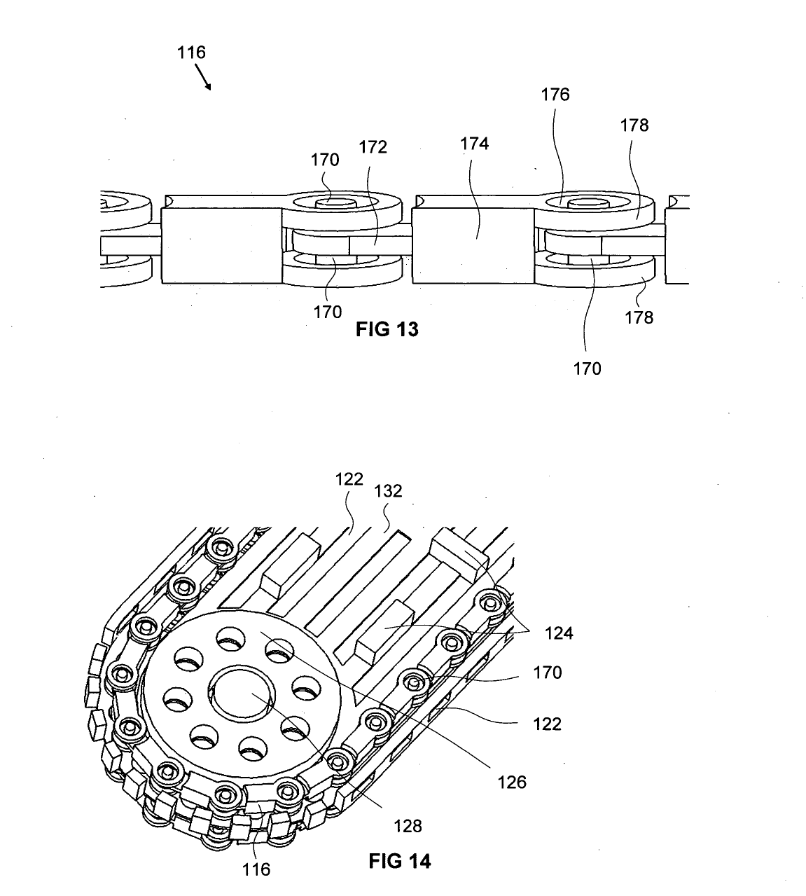

In FIG. 13, a portion of the upper housing chain 116 (which is identical to the lower housing chain) is seen in detail. It includes a set of identical links having a link body 174 at the center, a male extension 172 at one end, and two female extensions 178 at the other end. The male extension 172 is provided with pivot pins 170 extending from opposite surfaces. The clearance between the holes 176 in the female extensions 178 and the pivot pins 170 is normally set to the minimum in-layer feature size (e.g., 20 μm). More detail about minimum in-layer feature size associated with electrochemical fabrication is discussed in U.S. patent application Ser. No. 10/949,744, filed Sep. 24, 2004, by Cohen, and entitled “Three-Dimensional Structures Having Feature Sizes Smaller Than a Minimum Feature Size and Methods for Fabricating” and in U.S. patent application Ser. No. ______ (Microfabrica Docket No.P-US158-A-MF, filed May 26, 2006, by Cohen and entitled “Micro-Turbines, Roller Bearings, Bushings, and Design of Hollow Closed Structures and Fabrication Methods for Creating Such Structures”. These referenced applications also provide techniques for forming structures with smaller feature sizes (openings or voids and structural elements) that may be combined with the teachings herein to form instruments having smaller features or tighter feature tolerances. Each of the above noted applications is hereby incorporated herein by reference as if set forth in full.

When the chain ends are first pulled, the chain stretches slightly while eliminating the clearance between the pin and one side of the hole. In alternative configurations, the pin may be attached to the two outer arms while a single central ring structure attaches a next link. Other chain configurations are possible in other embodiments including chain configurations with multiple male and female connecting elements that join links together.

FIG. 14 shows a sectional view in which the upper compartment 134 of the upper housing 110 has been removed making the upper side of the lower compartment 132 visible. Visible is the chain 116 wrapped around a pulley 132 which is free to turn on a shaft 128. In some embodiments, the pulley need not necessarily rotate, as the chain may be allowed to slip over the pulley surface when pulled. If pulley motion is desired, slippage can be eliminated by shaping the pulley in the form of a sprocket and providing features on the chain links to receive the sprocket teeth. Instead of a bushing-like structure shown here, a roller or other bearing may be used to reduce friction between pulley 126 and 128 and shaft. FIG. 14 also shows structural posts which connect the floor and the roof of the lower (chain) compartment between etch holes to provide added structural rigidity (if needed).

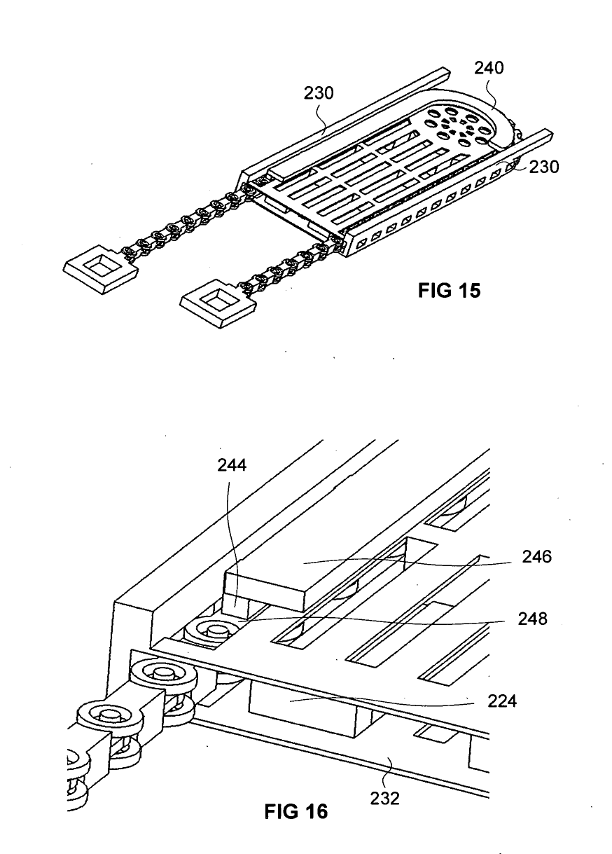

FIG. 15 shows a sectional perspective view in which the entire top housing 110 and the roof of the upper compartment 234 of lower housing 210 has been removed. A hook tool 240 can be seen inside the upper compartment 234 of the housing 210, in its retracted. In some embodiments, the tool may be in the retracted position during fabrication while in other embodiments it may be fully or partially extended during fabrication. The hook shown in FIG. 15 is merely an example of a potentially useful tool. In some embodiments, such a hook or other structure may be electrically isolated from the rest of the instrument. One or more Hook-like or finger-like shapes (as well as others) may be useful as electrical cutting or cauterizing tools for tissue and thus may appropriately isolated via built in dielectric materials and connected to electrical power supplies. The sides of the upper compartment form guide rails 230 for the tool as it moves, and may extend out past the curve of the lower compartment as shown.

FIG. 16 shows a more detailed view of a portion of FIG. 15. A slot 248 in the roof of the lower compartment 232 allows one or more tool attachment posts 244 to connect the base 246 of the tool 240 to one or more links of the chain while allowing motion along the instrument axis. If the attachment post is longer than one standard link or if there are multiple posts, in some embodiments, the standard links may be replaced by a ‘fused link’ which includes an elongated continuous stretch of metal. In some embodiments the design may be such that when fully extended, the link or links that hold the tool never travels past the pulley-chain contact point, while in other embodiments further travel may be allowed or even desired

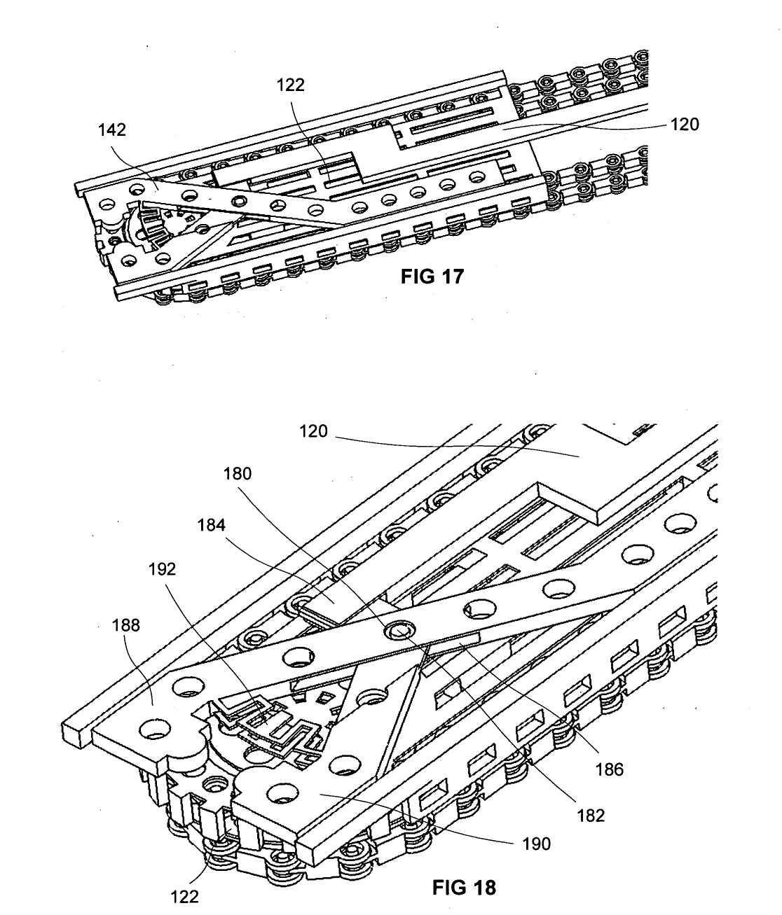

FIG. 17 shows a perspective sectional view in which the roof of the top housing 134 has been removed along with the bottom portion of the lower housing 132. Visible is the upper tool 140, which in the embodiment shown, is a forceps capable of being actuated by tension applied to the actuator tab 118. As shown in the detail of FIG. 18, the forceps 140 has a fixed jaw 188 and moveable jaw 190. The base of the fixed jaw 188 is joined to an attachment post (not shown) like that discussed above for the hook 240, thus allowing the forceps tool 140 to be extended or retracted in similar fashion by pulling on the appropriate chain end. The fixed jaw 188 has a hole 180 to receive a pivot pin 182 in the moveable jaw 190, and a slot 186 which allows for rotation of the moveable jaw 190 relative to the fixed jaw 188. The moveable jaw 190 is closed by pulling on its proximal end using the forceps actuator arm 120 and tab 118, whose distal end 184 has a pin that pivots in a hole (neither pin nor hole is visible in figures) of the moveable jaw. In the illustrated embodiment, when the jaw is released, the return spring 192 forces the jaws apart. In other embodiments, the jaws may be forced open by applying a compression force to tool arm 120. In some embodiments, the pulling on the forceps actuator could cause the entire forceps to be retracted into its housing, as such, in some embodiment it may be necessary to either simultaneously pull on the end of the upper housing chain 116 that extends the forceps tool 140, or else to lock it temporarily (or permanently) into an extended position before actuating the jaw.

In some alternative embodiments, the number of actuation chains/cables, may be reduced by providing a spring that pulls the tools back into the housings (or alternatively, extends them if already in) once the chain is released. This could be a linearly-acting spring or perhaps a clock-type spring attached to the end of the spring or to the pulley (this would require a no-slip condition between chain and pulley: e.g. the pulley could have a sprocket and the chain could be modified to accept the sprocket teeth).

In some embodiments, multiple housing modules may be used to actuate a single tool (e.g. part of a tool may be extended from one module while another portion of the tool may extend from another module)

The techniques disclosed explicitly herein may benefit by combining them with the techniques disclosed in U.S. patent application Ser. No. 10/841,272 filed May 7, 2004 by Adam Cohen et al. and entitled “Methods and Apparatus for Forming Multi-Layer Structures Using Adhered Masks”. This referenced application is incorporated herein by reference as if set forth in full herein. This referenced application teaches various electrochemical fabrication methods and apparatus for producing multi-layer structures from a plurality of layers of deposited materials where adhered masks are used in selective patterning operations.

The techniques disclosed explicitly herein may benefit by combining them with the techniques disclosed in U.S. patent application Ser. No. 10/697,597 filed on Oct. 29, 2003 by Michael S. Lockard et al. and entitled “EFAB Methods and Apparatus Including Spray Metal or Powder Coating Processes”. This referenced application is incorporated herein by reference as if set forth in full herein. This referenced application teaches various techniques for forming structures via a combined electrochemical fabrication process and a thermal spraying process or powder deposition processes. In some embodiments, selective deposition occurs via masking processes (e.g. a contact masking process or adhered mask process) and thermal spraying or powder deposition is used in blanket deposition processes to fill in voids left by the selective deposition processes. In other embodiments, after selective deposition of a first material, a second material is blanket deposited to fill in the voids, the two depositions are planarized to a common level and then a portion of the first or second materials is removed (e.g. by etching) and a third material is sprayed into the voids left by the etching operation. In both types of embodiments the resulting depositions are planarized to a desired layer thickness in preparation for adding additional layers.

The techniques disclosed explicitly herein may benefit by combining them with various elements of the dielectric substrate on and/or dielectric incorporation techniques disclosed in the following patent applications (1) U.S. Patent Application Ser. Nos. 60/534,184 filed Dec. 31, 2003 and 11/029,216 filed Jan. 3, 2005 both by Adam L. Cohen et al and entitled “Electrochemical Fabrication Methods Using Dielectric Substrates and/or Incorporating Dielectric Materials”; (2) U.S. Patent Application Ser. No. 60/533,932 filed Dec. 31, 2003 by Adam L. Cohen et al. and entitled “Electrochemical Fabrication Methods Using Dielectric Substrates and/or Incorporating Dielectric Materials”; (3) U.S. Patent Application Ser. No. 60/534,157, which is entitled “Electrochemical Fabrication Methods Incorporating Dielectric Materials”, filed Dec. 31, 2003 by Lockard et al; and (4) U.S. Patent Application Ser. No. 60/533,895 filed Dec. 31, 2003 by Lembrikov et al, and entitled “Electrochemical Fabrication Method for Producing Multi-layer Three-Dimensional Structures on a Porous Dielectric”. These applications are hereby incorporated herein by reference as if set forth in full.

The patent applications and patents set forth below are hereby incorporated by reference herein as if set forth in full. The teachings in these incorporated applications can be combined with the teachings of the instant application in many ways: For example, enhanced methods of producing structures may be derived from some combinations of teachings, enhanced structures may be obtainable, enhanced instruments or apparatus may be derived, and the like.

| US Pat App No, Filing Date | |
|---|---|
| US App Pub No, Pub Date | Inventor, Title |
| 09/493,496 - Jan. 28, 2000 | Cohen, “Method For Electrochemical Fabrication” |
| 10/677,556 - Oct. 1, 2003 | Cohen, “Monolithic Structures Including Alignment |
| | and/or Retention Fixtures for Accepting Components” |
| 10/830,262 - Apr. 21, 2004 | Cohen, “Methods of Reducing Interlayer |
| | Discontinuities in Electrochemically Fabricated Three- |
| | Dimensional Structures” |
| 10/271,574 - Oct. 15, 2002 | Cohen, “Methods of and Apparatus for Making High |
| 2003-0127336A - Jul. 10, 2003 | Aspect Ratio Microelectromechanical Structures” |
| 10/697,597 - Dec. 20, 2002 | Lockard, “EFAB Methods and Apparatus Including |
| | Spray Metal or Powder Coating Processes” |
| 10/677,498 - Oct. 1, 2003 | Cohen, “Multi-cell Masks and Methods and Apparatus |
| | for Using Such Masks To Form Three-Dimensional |
| | Structures” |
| 10/724,513 - Nov. 26, 2003 | Cohen, “Non-Conformable Masks and Methods and |
| | Apparatus for Forming Three-Dimensional Structures” |
| 10/607,931- Jun. 27, 2003 | Brown, “Miniature RF and Microwave Components and |
| | Methods for Fabricating Such Components” |
| 10/841,100 - May 7, 2004 | Cohen, “Electrochemical Fabrication Methods |
| | Including Use of Surface Treatments to Reduce |
| | Overplating and/or Planarization During Formation of |
| | Multi-layer Three-Dimensional Structures” |
| 10/387,958 - Mar. 13, 2003 | Cohen, “Electrochemical Fabrication Method and |
| 2003-022168A - Dec. 4, 2003 | Application for Producing Three-Dimensional |
| | Structures Having Improved Surface Finish” |
| 10/434,494 - May 7, 2003 | Zhang, “Methods and Apparatus for Monitoring |
| 2004-0000489A - Jan. 1, 2004 | Deposition Quality During Conformable Contact Mask |
| | Plating Operations” |
| 10/434,295 - May 7, 2003 | Cohen, “Method of and Apparatus for Forming Three- |
| 2004-0004001A - Jan. 8, 2004 | Dimensional Structures Integral With Semiconductor |
| | Based Circuitry” |
| 10/434,315 - May 7, 2003 | Bang, “Methods of and Apparatus for Molding |
| 2003-0234179 A - Dec. 25, 2003 | Structures Using Sacrificial Metal Patterns” |
| 10/434,103 - May 7, 2004 | Cohen, “Electrochemically Fabricated Hermetically |
| 2004-0020782A - Feb. 5, 2004 | Sealed Microstructures and Methods of and Apparatus |
| | for Producing Such Structures” |
| 10/841,006 - May 7, 2004 | Thompson, “Electrochemically Fabricated Structures |
| | Having Dielectric or Active Bases and Methods of and |
| | Apparatus for Producing Such Structures” |
| 10/434,519 - May 7, 2003 | Smalley, “Methods of and Apparatus for |
| 2004-0007470A - Jan. 15, 2004 | Electrochemically Fabricating Structures Via Interlaced |
| | Layers or Via Selective Etching and Filling of Voids” |
| 10/724,515 - Nov. 26, 2003 | Cohen, “Method for Electrochemically Forming |
| | Structures Including Non-Parallel Mating of Contact |
| | Masks and Substrates” |
| 10/841,347 - May 7, 2004 | Cohen, “Multi-step Release Method for |
| | Electrochemically Fabricated Structures” |
| 60/533,947 - Dec. 31, 2003 | Kumar, “Probe Arrays and Method for Making” |
| 60/534,183 - Dec. 31, 2003 | Cohen, “Method and Apparatus for Maintaining |
| | Parallelism of Layers and/or Achieving Desired |
| | Thicknesses of Layers During the Electrochemical |
| | Fabrication of Structures” |
| 11/029,220 - Jan. 3, 2005 | Frodis, “Method And Apparatus for Maintaining |
| | Parallelism of Layers and/or Achieving Desired |
| | Thicknesses of Layer During the Electrochemical |
| | Fabrication of Structures” |
| 60/695,328 - Jun. 26, 2005 | Cohen, “Electrochemical Fabrication Processes |
| | Incorporating Non-Platable Materials” |

Various other embodiments of the present invention exist. Some of these embodiments may be based on a combination of the teachings herein with various teachings incorporated herein by reference. Some embodiments may not use any blanket deposition process and/or they may not use a planarization process. Some embodiments may use selective deposition processes or blanket deposition processes on some layers that are not electrodeposition processes. Some embodiments, for example, may use nickel, nickel titanium, nickel cobalt, titanium, stainless steel, gold, copper, tin, silver, zinc, solder, various alloys of these and other materials as structural materials while other embodiments may use different materials. Some embodiments, for example, may use copper, tin, zinc, solder or other materials as sacrificial materials. Some embodiments may remove a sacrificial material while other embodiments may not. Some embodiments may use photoresist, polyimide, glass, ceramics, other polymers, and the like as dielectric structural materials

In view of the teachings herein, many further embodiments, alternatives in design and uses of the instant invention will be apparent to those of skill in the art. As such, it is not intended that the invention be limited to the particular illustrative embodiments, alternatives, and uses described above but instead that it be solely limited by th claims presented hereafter.

I claim:

1. An micro-scale or mesoscale instrument containing multiple independently deployable tools comprising:
a. a first tool having a first functionality;
b. a second tool having a second functionality;
c. at least one housing, having at least one proximal end and at least one distal end, for holding the first and second tools when in a retracted state a housing; and
d. at least one mechanism extending from the proximal end of the at least one housing to which a tensional force may be exerted to cause deployment of at least one of the first or second tool from the at least one housing.

2. The instrument of claim 1 having a plurality of microscale structural features.

3. The instrument of claim 1 additionally comprising:
e. at least one mechanism capable of retracting at least one of the first or second tools back into the at least one housing.

4. The instrument of claim 1 wherein the at least one housing comprises at least two housings.

5. The instrument of claim 1 wherein the at least one mechanism comprises at least one chain.

6. The instrument of claim 5 wherein the at least one mechanism comprises at least two mechanisms and each mechanism comprises a chain.

7. The instrument of claim 1 which is suitable for use in a minimally invasive surgical procedure.

8. The instrument of claim 5 wherein the at least one mechanism comprises a pulley around which the at least one chain wraps.

9. The instrument of claim 1 additionally comprising an actuating mechanism for actuating at least one tool when the tool is deployed.

10. The instrument of claim 1 wherein at least one of the first or second tools is deployable from the at least one housing via substantially linear extension from the distal end of the at least one housing.

11. The instrument of claim 10 wherein the at least one of the first or second tools is deploy along a line that is parallel to an axis of the instrument.

12. A micro-scale or mesoscale surgical instrument containing multiple independently deployable tools comprising:
a. a first module comprising a first housing and a first tool having a first functionality which is deployable from the first housing; and
b. a second module comprising a second housing and a second tool having a second functionality which is deployable form the second housing,
wherein the first module and second module are stacked and fixed together.

13. The instrument of claim 12 abovewhich is a minimally invasive surgical instrument.

14. A micro-scale or mesoscale instrument, comprising:
a. a module comprising a housing and a tool having a first functionality which is deployable from a distal end of the housing; and
b. a bidirectional chain connected to the tool within the housing and having two ends that extend from the housing,
wherein pulling on a first end of the chain causes a retracted tool to be deployed from the housing; and
wherein pulling on a second end of the chain, which is different from the first end, causes a deployed tool to be retracted into the housing.

15. The instrument of claim Error! Reference source not found.which is a minimally invasive surgical instrument.

16. A micro-scale or mesoscale instrument, comprising:
a. a module comprising a housing and a tool having a first functionality which is deployable from a distal end of the housing; and
b. a mechanism for deploying the tool from the housing,
wherein the tool is deployable from the housing along a non-pivoting path.

17. The instrument of claim 16 wherein the non-pivoting path is substantially linear path that is parallel to an axis of the instrument.
